(19) United States
(12) Patent Application Publication
(10) Pub. No.: US 2016/0354130 A1
(43) Pub. Date: Dec. 8, 2016

(54) BONE TAMP AND METHOD OF USE

(71) Applicant: Kyphon SÀRL, Neuchatel (CH)

(72) Inventors: Bryan Auyoung, Santa Clara, CA (US); Jusong Xia, Collierville, TN (US)

(21) Appl. No.: 14/728,183

(22) Filed: Jun. 2, 2015

(51) Int. Cl. A61B 17/88

(52) U.S. Cl. CPC A61B 17/8855; A61B 17/8841; A61B 17/8805

(57) ABSTRACT

A surgical system and method for treatment of a bone defect are provided. The system includes a bone tamp that includes an outer shaft defining a first lumen and a second lumen. A first expandable body is coupled to the outer shaft. The first expandable body is in communication with the first lumen such that an inflation material may be moved through the first lumen and into the first expandable body. An inner shaft is movably positioned within the second lumen. The inner shaft defines a third lumen. A second expandable body is coupled to the inner shaft. The second expandable body is in communication with the third lumen such that an inflation material may be moved through the third lumen and into the second expandable body.

### TECHNICAL FIELD

The present disclosure generally relates to medical devices for the treatment of bone disorders, and more particularly to a surgical system and method employing a bone tamp to provide provisional height restoration to a bone while the bone tamp creates a cavity in the bone and fills the cavity with a material, such as, for example, bone cement to permanently restore the height of the bone.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Many spinal disorders may be treated by restoring the height of vertebrae and/or reducing fractures or other defects in the vertebrae. In some instances, the treatment is done percutaneously using inflatable bone tamps (IBTs) with or without other instrumentation. Indeed, in an effort to more effectively and directly treat vertebral compression fractures and fractures in other bones, minimally invasive techniques such as vertebroplasty and, subsequently, kyphoplasty, have been developed. Vertebroplasty involves creating a cavity in a fractured, weakened, or diseased vertebral body using an IBT. The IBT is typically removed after the cavity is created. A flowable reinforcing material, such as, for example, bone cement (e.g., polymethylmethacrylate (PMMA)) is then injected into the cavity using another instrument, such as, for example, a bone cement delivery tool that is inserted into the patient after the IBT is removed. Shortly after injection, the bone cement hardens or polymerizes, desirably supporting the vertebral body internally, alleviating pain and preventing further collapse of the injected vertebral body. In some instances, multiple cavities need to be created and filled, thus requiring that the IBT and the bone cement delivery tool each be inserted into and removed from the patient multiple times during a given procedure. However, the repeated insertion and removal of multiple tools in and out of the patient creates undue risk of injury, increases the length and/or cost of a procedure and likely increases recovery time. Furthermore, because the IBT and bone cement delivery tool are typically not present in patient simultaneously, the IBT is unable to provisionally restore the height of the bone while the bone cement is injected into the cavity and/or while the bone cement hardens or polymerizes, thus resulting in a loss of at least some of the restored height. This disclosure describes an improvement over these prior art technologies.

### SUMMARY

In one embodiment, a system for performing a surgical procedure is provided, in accordance with the principles of the present disclosure. The system includes a bone tamp having an outer shaft that defines a first lumen and a second lumen. A first expandable body is coupled to the outer shaft. The first expandable body is in communication with the first lumen such that an inflation material may be moved through the first lumen and into the first expandable body. An inner shaft is movably positioned within the second lumen. The inner shaft defines a third lumen. A second expandable body is coupled to the inner shaft. The second expandable body is in communication with the third lumen such that an inflation material may be moved through the third lumen and into the second expandable body.

In one embodiment, a system for performing a surgical procedure is provided, in accordance with the principles of the present disclosure. The system includes a bone tamp having an outer shaft that defines a first lumen and a second lumen. A first expandable body is coupled to the outer shaft. The first expandable body is in communication with the first lumen such that an inflation material may be moved through the first lumen and into the first expandable body. An inner shaft is movably positioned within the second lumen. The inner shaft defines a third lumen. A second expandable body is coupled to the inner shaft. The second expandable body is in communication with the third lumen such that an inflation material may be moved through the third lumen and into the second expandable body. The system further includes a bone cement delivery device and bone cement. The inner shaft is removably positioned within the second lumen and the second lumen is configured for disposal of at least a portion of the bone cement delivery device such that the bone delivery device can deliver the bone cement from a distal end of the inner shaft.

In one embodiment, a system for performing a surgical procedure is provided, in accordance with the principles of the present disclosure. The system includes a bone tamp having an outer shaft defining a first lumen and a second lumen. The first lumen surrounds the second lumen such that the first lumen is isolated from the second lumen. A first expandable body is coupled to the outer shaft. The first expandable body is in communication with the first lumen such that an inflation material may be moved through the first lumen and into the first expandable body. An inner shaft is slidably positioned within the second lumen. The inner shaft defines a third lumen. The inner shaft is removable from the outer shaft. A second expandable body is coupled to the inner shaft. The second expandable body is in communication with the third lumen such that an inflation material may be moved through the third lumen and into the second expandable body. The system further includes a bone cement delivery device and bone cement. The inner shaft is removably positioned within the second lumen and the second lumen is configured to carry the bone cement out of the distal end of the second lumen. The first expandable body is inflatable independent of the second expandable body. In one embodiment, the first expandable body has a surface area that is greater than that of the second expandable body when the first and second expandable bodies are each in an expanded configuration. Alternative configures of the expandable bodies may also be used in accordance with the principles of the present disclosure.

In one embodiment, a method for repairing a bone defect is provided, in accordance with the principles of the present disclosure. The method comprises providing a bone tamp having an outer shaft that defines a first lumen and a second lumen. A first expandable body is coupled to the outer shaft. The first expandable body is in communication with the first lumen such that an inflation material may be moved through the first lumen and into the first expandable body. An inner shaft is movably positioned within the second lumen. The inner shaft defines a third lumen. A second expandable body is coupled to the inner shaft. The second expandable body is in communication with the third lumen such that an inflation material may be moved through the third lumen and into the second expandable body. The system further includes a bone cement delivery device and bone cement. The inner shaft is removably positioned within the second lumen and the second lumen is configured for disposal of at least a portion of the bone cement delivery device such that the bone cement delivery device can deliver the bone cement from a distal end of the inner shaft.

The method further comprises positioning the first expandable body within a bony structure and injecting an inflation material through the first lumen and into the first expandable body to move the first expandable body from an unexpanded configuration to an expanded configuration. The second expandable body is then positioned within the bony structure and an inflation material is injected through the third lumen and into the second expandable body to move the second expandable body from an unexpanded configuration to an expanded configuration such that the second expandable body creates a void within the bony structure. The inflation material is withdrawn from the second expandable body to move the second expandable body from the expanded configuration to an unexpanded configuration. The inner shaft is removed from the second lumen. With the first expandable body still in the expanded configuration so as to maintain the restored height, bone cement is injected through the second lumen to fill at least part of the void. Once the cement is at least partially hardened, the first expendable balloon is deflated and additional cement is injected through the second lumen to fill the remaining portion of the void.

### BRIEF DESCRIPTION OF THE DRAWINGS

The present disclosure will become more readily apparent from the specific description accompanied by the following drawings, in which:

FIG. 1 is a side view of components a surgical system in accordance with the principles of the present disclosure;

FIG. 2 is a side view of components shown in FIG. 1, with parts separated;

FIG. 3 is a cross sectional view of one of the components shown in FIG. 1 at Detail A in FIG. 2;

FIG. 4 is a cross sectional view of one of the components shown in FIG. 1 at Detail B in FIG. 2;

FIG. 5 is a cross sectional view of one embodiment of one of the components shown in FIG. 1;

FIG. 5A is a perspective view, in part phantom, of a portion of components shown in FIG. 1;

FIG. 5B is a perspective view, in part phantom, of a portion of components shown in FIG. 1;

FIG. 6 is a cross sectional view of one embodiment of one of the components shown in FIG. 1;

FIG. 6A is a perspective view, in part phantom, of a portion of components shown in FIG. 1;

FIG. 7 is a cross sectional view of one of the components shown in FIG. 1 at Detail C in FIG. 2;

FIG. 8 is a side view of a component of the system surgical in accordance with the principles of the present disclosure;

FIG. 9 is a side view of one of the components shown in FIG. 1;

FIG. 10 is a side view of the components shown in FIG. 1;

FIG. 11 is a side view of the components shown in FIG. 1;

FIG. 12 is a side view of the components shown in FIG. 1;

FIG. 13 is a perspective view, in part phantom, of components a surgical system in accordance with the principles of the present disclosure; and

FIG. 14 is an end view of components of the system shown in FIG. 13.

Like reference numerals indicate similar parts throughout the figures.

### DETAILED DESCRIPTION

The exemplary embodiments of the surgical system and related methods of use disclosed are discussed in terms of medical devices for the treatment of bone disorders and more particularly, in terms of a surgical system and method for bone repair. In some embodiments, the surgical system and method permit a unilateral approach to balloon kyphoplasty by allowing for cement introduction while a balloon remains inflated to maintain a restored height of the bone. It is envisioned that the surgical system and method may be employed in applications such as for correction of fractures, depressions and breaks. For example, the surgical system and method can comprise IBTs presenting a straight or an angled surface to restore vertebra height or to repair bones and the IBT can provisionally maintain the restored height of the bone while a cavity in the bone made by the same or a different IBT is filled using bone cement to permanently maintain the restored height of the bone.

In one embodiment, the system includes a tandem IBT concept in a unique design that is intended for a unilateral approach. In particular, the unique design features two independently controlled IBTs that can be used in a surgical procedure, such as, for example, in a procedure to reduce a vertebral compression fracture. The two independently controlled IBTs comprise a distal IBT and a proximal IBT. The distal IBT is intended to travel through a lumen in the proximal IBT. Both the proximal IBT and the distal IBT may be introduced into a surgical site, such as, for example, a vertebral body simultaneously. Both the proximal IBT and the distal IBT may each be inserted introduced into the surgical site when in an uninflated or unexpanded configuration. After the proximal IBT and the distal IBT are positioned adjacent to the surgical site, the proximal IBT and the distal IBT may be inflated to move the proximal IBT and the distal IBT from the uninflated or unexpanded configurations to inflated or expanded configurations. In some embodiments, moving the proximal IBT and the distal IBT from the uninflated or unexpanded configurations to the inflated or expanded configurations creates a void, reduces a fracture and/or obtains at least some height restoration.

The distal IBT may be moved from the inflated or expanded configuration to the uninflated or unexpanded configuration by deflating the distal IBT while the proximal IBT remains in the inflated or expanded configuration. The distal IBT may be withdrawn from the surgical site through the proximal IBT. The proximal IBT remains in the inflated or expanded configuration following withdrawal of the distal IBT to maintain height restoration while a filler material, such as, for example, bone cement is introduced into the void created by the distal IBT. In some embodiments, the bone cement is introduced through a lumen in the proximal IBT that was previously occupied by the distal IBT. Once the void created by the distal balloon is at least partially filled with filler, the filler is allowed to at least partially harden before the proximal balloon is deflated. Keeping the proximal balloon inflated while the filler is allowed to harden helps maintain the overall restored height.

In some embodiments, the proximal IBT includes a balloon that is resistant to bone cement, such as, for example, PMMA. In some embodiments, the proximal IBT includes a first balloon and the distal IBT includes a second balloon, wherein the first and second balloons are formed from the same material. In some embodiments, the proximal IBT includes a first balloon and the distal IBT includes a second balloon, wherein the first balloon is formed from an entirely different material than the second balloon. In one embodiment, the first balloon comprises nylon and the second balloon comprises polyurethane.

It is contemplated that one or all of the components of the surgical system may be disposable, peel-pack, pre-packed sterile devices. One or all of the components of the surgical system may be reusable. The surgical system may be configured as a kit with multiple sized and configured components.

It is envisioned that the present disclosure may be employed to treat bones, and in particular extremity bones such as vertebrae and the calcaneus. It should be understood that the present principles are applicable to any bone structures, including but not limited to bones of the spine, legs, feet, arms, etc. It is contemplated that the present disclosure may be employed with other osteal and bone related applications, including those associated with diagnostics and therapeutics. It is further contemplated that the disclosed surgical system and methods may alternatively be employed in a surgical treatment with a patient in a prone or supine position, and/or employ various surgical approaches, including anterior, posterior, posterior mid-line, direct lateral, postero-lateral, antero-lateral, etc. approaches in the calcaneus, spine or other body regions. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” comprise the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may comprise administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can comprise inhibiting the disease, e.g., arresting its development, or relieving the disease, e.g., causing regression of the disease. For example, treatment can comprise reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

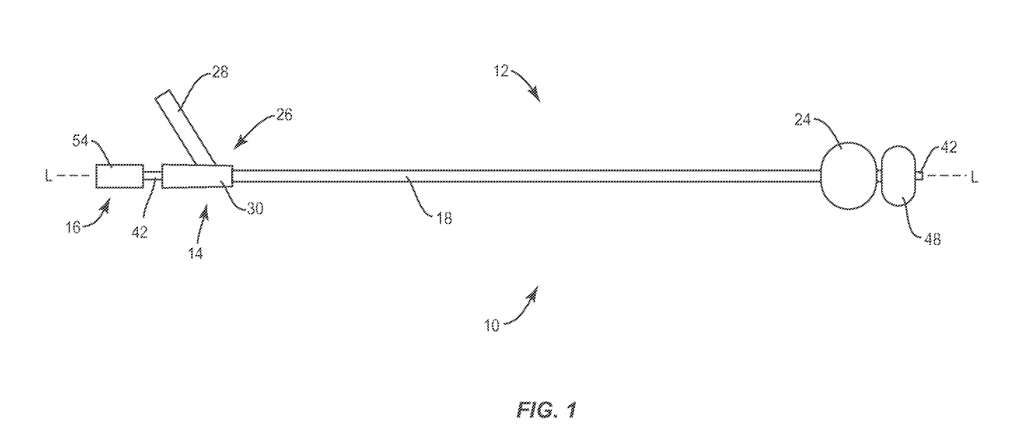

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-14, there are illustrated components of a surgical system, such as, for example, a system 10 and embodiments in accordance with the principles of the present disclosure.

The components of system 10 can be fabricated from biologically acceptable materials suitable for medical applications, including metals, synthetic polymers, ceramics and bone material and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), nylon, stiff nylon or other high stiffness polymers, ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or comprise fastening elements and/or instruments, as described herein.

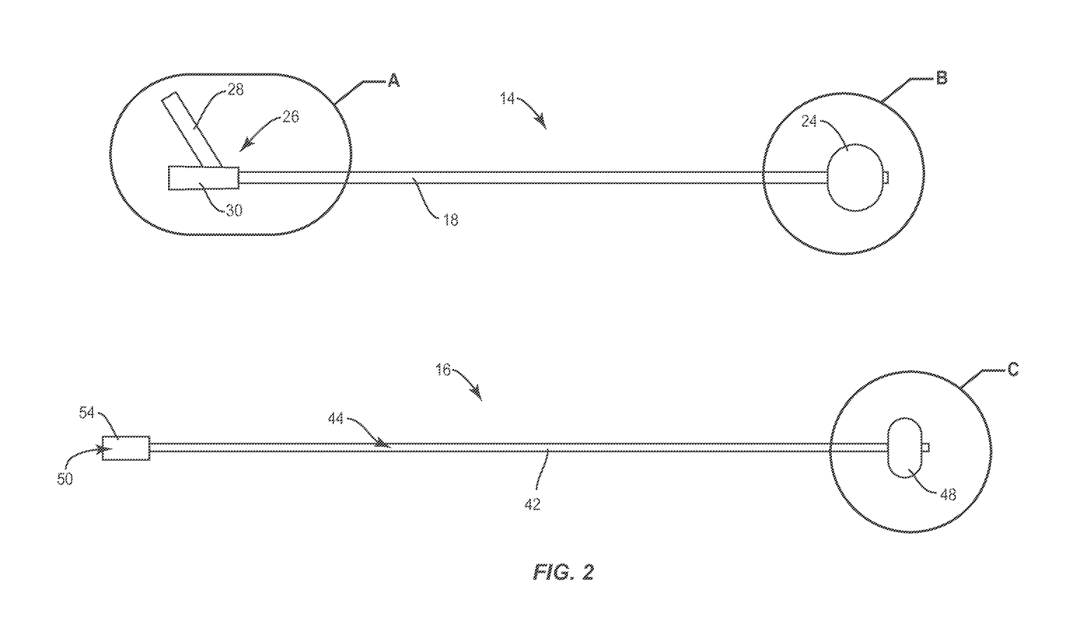

System 10 comprises a tandem inflatable bone tamp 12 configured to permit a unilateral approach to balloon kyphoplasty, while allowing for cement introduction while a balloon remains inflated to maintain height restoration, as will be discussed. Tandem inflatable bone tamp 12 comprises a first inflatable bone tamp 14 and a second inflatable bone tamp 16. Second inflatable bone tamp 16 is shown extending through first inflatable bone tamp 14 in FIG. 1. First inflatable bone tamp 14 is shown separated from second inflatable bone tamp 16 in FIG. 2. First inflatable bone tamp 14 and a second inflatable bone tamp 16 are independently controlled. That is, first inflatable bone tamp 14 may be moved from an uninflated or unexpanded configuration to an inflated or expanded configuration independently of second inflatable bone tamp 16 and second inflatable bone tamp 16 may be moved from an uninflated or unexpanded configuration to an inflated or expanded configuration independently of first inflatable bone tamp 14, as discussed herein.

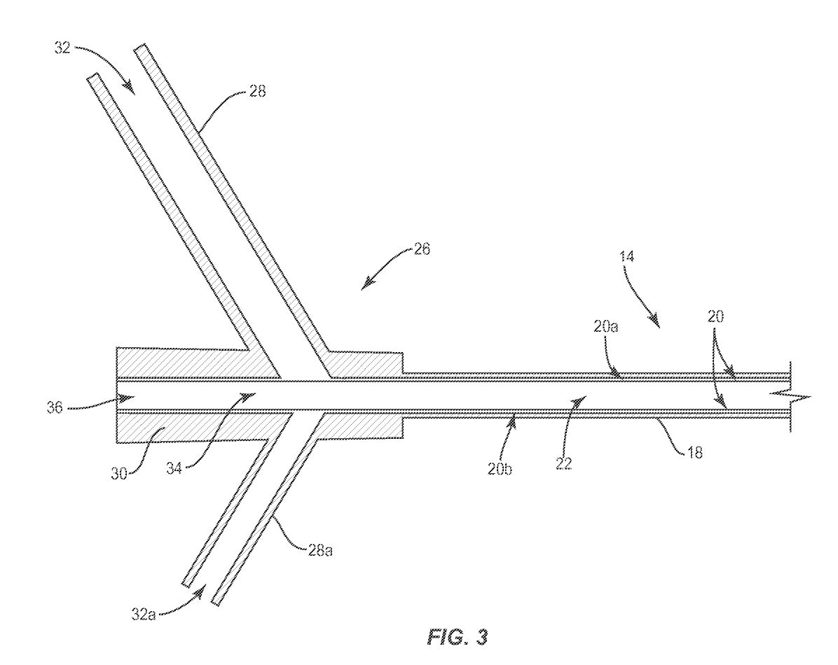

First inflatable bone tamp 14 comprises an outer shaft 18 that defines a first lumen 20 and a second lumen 22, as shown in FIGS. 3-6, for example. First lumen 20 is configured to deliver an inflation material, such as, for example, pressurized air or saline into a first expandable body, such as, for example, a balloon 24 of first inflatable bone tamp 14 that is coupled to outer shaft 18 to move balloon 24 from an uninflated or unexpanded configuration to an inflated or expanded configuration. Second lumen 22 is configured for disposal of second inflatable bone tamp 16. First lumen 20 is isolated from second lumen 22. That is, first lumen 20 is not in fluid communication with second lumen 22 such that a material in first lumen 20 will not enter second lumen 22, and vice versa. First lumen 20 and second lumen 22 each extend continuously from a proximal end of outer shaft 18 to a distal end of outer shaft 18.

In some embodiments, outer shaft 18 comprises a flexible material such as, for example, any of the flexible materials discussed above, to allow outer shaft 18 to bend without breaking. Providing flexibility to outer shaft 18 may be beneficial to deliver and/or position balloons of first inflatable bone tamp 14 or second inflatable bone tamp 16 to a selected location, such as, for example, a surgical site, as discussed herein, where outer shaft 18 may need to be bent to navigate around an obstruction. In some embodiments, outer shaft 18 comprises a rigid material to provide strength to outer shaft 18 such that outer shaft 18 cannot bend without breaking. In some embodiments, outer shaft 18 may be transparent or translucent to allow an inflation material and/or second inflatable bone tamp 16 to be viewable through outer shaft 18 such that the location of the inflation material and/or second inflatable bone tamp 16 within outer shaft 18 may be identified visually.

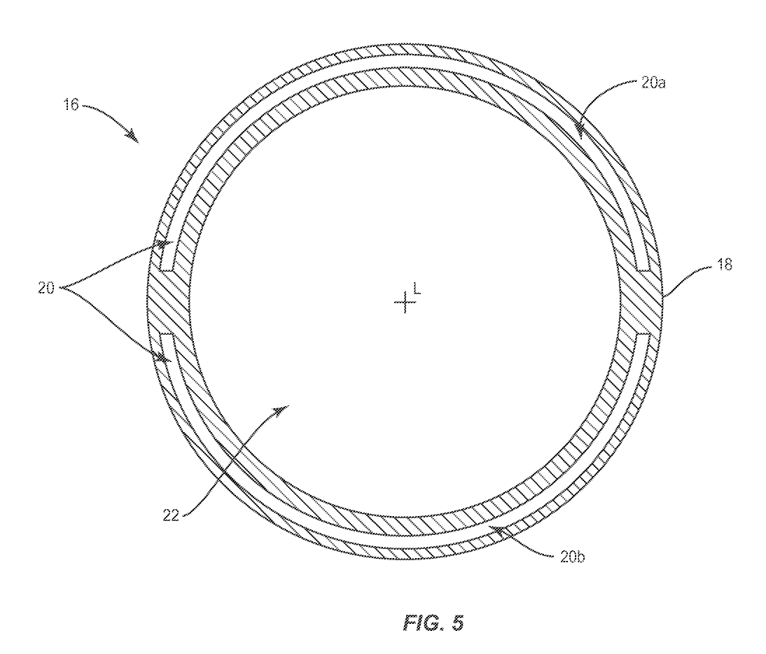

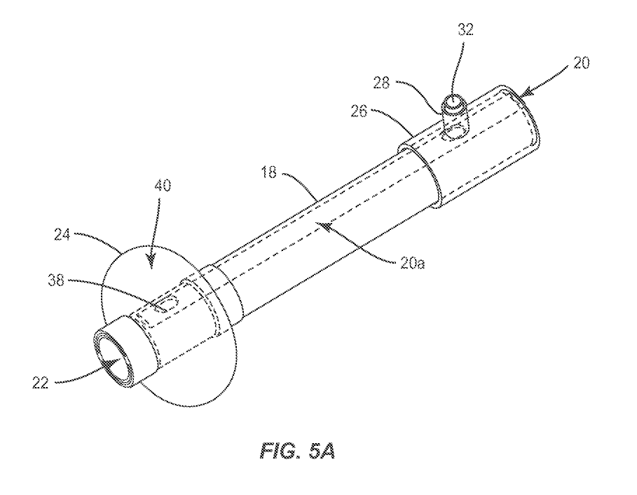

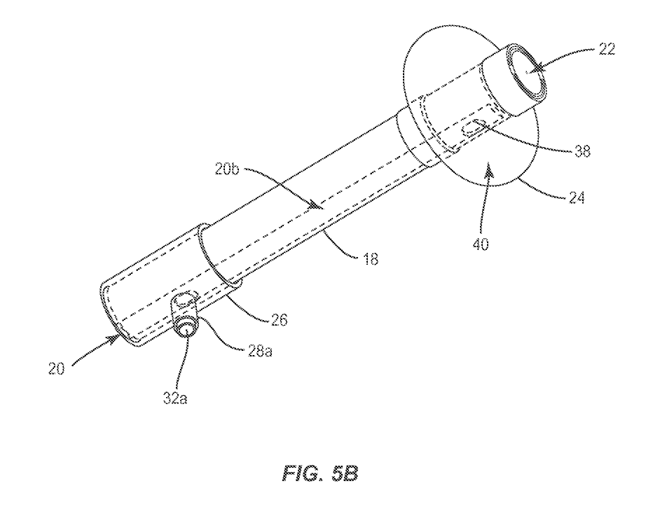

In one embodiment, shown in FIGS. 3-5b, first lumen 20 surrounds second lumen 22. First lumen 20 and second lumen 22 each extend parallel to a longitudinal axis L of outer shaft 18, as shown in FIG. 5. In the embodiment shown in FIGS. 3-5b, first lumen 20 is bifurcated such that first lumen 20 includes a first section 20a that is separated from a second section 20b. First section 20a and second section 20b each extend through a wall thickness of outer shaft 18 and each comprise a semi-circular cross sectional configuration. That is, first section 20a and second section 20b are both embedded between opposite inner and outer surfaces of outer shaft 18. Second lumen 22 has a circular cross sectional configuration and a maximum diameter that is greater than that of each of first section 20a and second section 20b. In some embodiments, first section 20a, second section 20b and/or second lumen 22 may have various cross section configurations, such as, for example, semicircular, oval, oblong, triangular, rectangular, square, polygonal, irregular, uniform, non-uniform, variable, tubular and/or tapered.

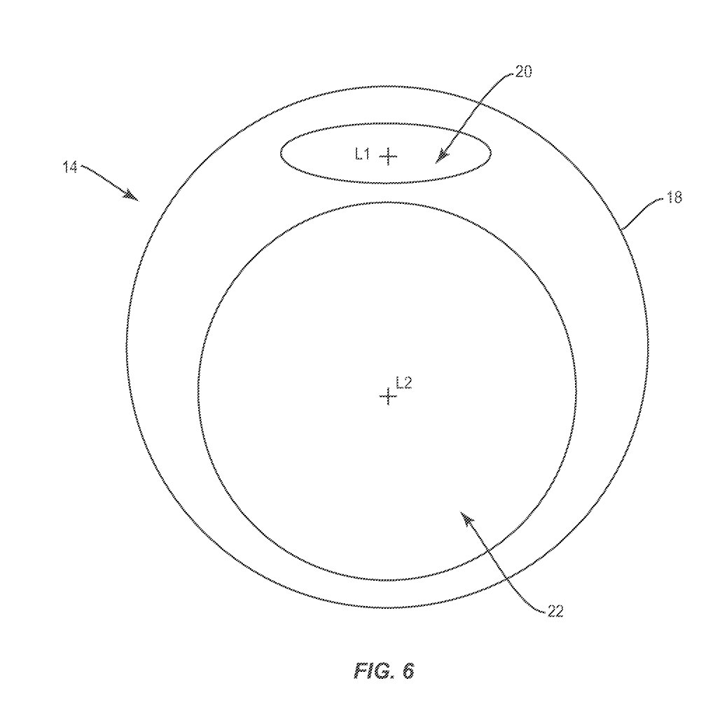

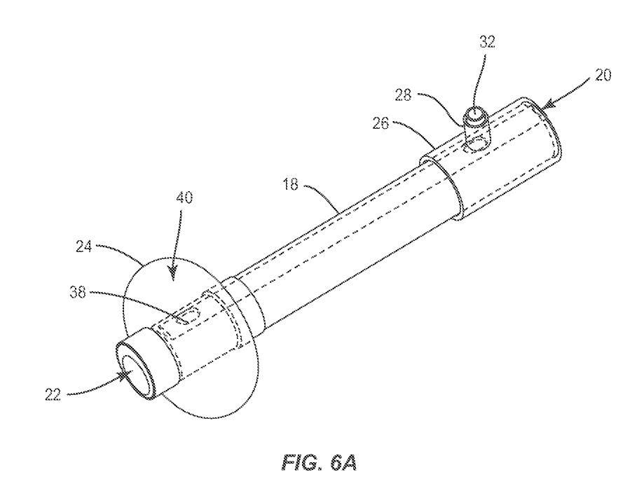

In one embodiment, shown in FIGS. 6 and 6A, first lumen 20 is coaxial with a longitudinal axis L1 and second lumen 22 is coaxial with a longitudinal axis L2 that is offset from longitudinal axis L2. First lumen 20 extends through a wall thickness of outer shaft 18 and second lumen 22 is defined by an inner surface of outer shaft 18. First lumen 20 has an oval cross sectional configuration and second lumen 22 has a circular cross sectional configuration. In some embodiments, first lumen 20 and/or second lumen 22 may have various cross section configurations, such as, for example, oval, oblong, triangular, rectangular, square, polygonal, irregular, uniform, non-uniform, variable, tubular and/or tapered.

A connector 26 is coupled to the proximal end of outer shaft 18. Connector 26 comprises a first extension 28 that extends transverse to longitudinal axis L and a second extension 30 that extends parallel to longitudinal axis L. As shown in FIGS. 3 and 6A, first extension 28 comprises a passageway 32 that is in communication with first lumen 20 of outer shaft 18. In the embodiment shown in FIGS. 3-5B, passageway 32 is in communication with first section 20a of first lumen 20. In the embodiment shown in FIGS. 3-5B, connector 26 comprises a third extension 28a that extends transverse to longitudinal axis L. Third extension 28a comprises a passageway 32a that is in communication with second section 20b of first lumen 20.

System 10 comprises a material source, such as, for example, a source of compressed air or saline. In the embodiment shown in FIGS. 6 and 6A, the material source is configured to be removably coupled to first extension 28 such that an inflation material can move from the material source, through passageway 32 and into first lumen 20 to move balloon 24 from an uninflated or unexpanded configuration to an inflated or expanded configuration, as discussed herein. In the embodiment shown in FIGS. 3-5B, the material source is configured to be removably coupled to first extension 28 such that an inflation material can move from the material source, through passageway 32 and into first section 20a of first lumen 20 to move balloon 24 from an uninflated or unexpanded configuration to an inflated or expanded configuration, as discussed herein. The same material source or a second material source may be removably coupled to third extension 28a such that an inflation material can move from the material source or the second material source, through passageway 32a and into second section 20b of first lumen 20 to move balloon 24 from an uninflated or unexpanded configuration to an inflated or expanded configuration, as discussed herein.

First lumen 20 is closed at a proximal end of second extension 30, as shown in FIG. 3, for example. That is, first lumen 20 does not extend through a proximal end surface of second extension 30 that extends transverse or perpendicular to longitudinal axis L to prevent inflation material from escaping first lumen 20 through second extension 30. In some embodiments, an inner surface and/or an outer surface of first extension 28 and/or third extension 28a comprises threads configured to mate with threads of the material source or the second material source to couple the material source to first extension 28 and/or to couple third extension 28a to the second material source. In some embodiments, the material source can be variously connected with first extension 28 and the material source or the second material source may be variously connected with third extension 28a, such as, for example, monolithic, integral connection, frictional engagement, threaded engagement, mutual grooves, screws, adhesive, nails, barbs and/or raised element.

Second extension 30 comprises a channel 34 that is in communication with second lumen 22, as shown in FIG. 3. Channel 34 is coaxial with longitudinal axis L. In some embodiments, channel 34 is continuous with second lumen 22. Channel 34 is isolated from first lumen 20. That is, first lumen 20 is not in fluid communication with channel 34 such that a material in first lumen 20 will not enter channel 34, and vice versa. A proximal end of second extension 30 comprises an opening 36 that is coaxial with longitudinal axis L. Opening 36 is in communication with channel 34 such that second inflatable bone tamp 16 may be inserted through opening 36 and channel 34 and into second lumen 22.

Balloon 24 extends between a proximal end and an opposite distal end. The proximal and distal ends of balloon 24 are each coupled to an outer surface of outer shaft 18. It is envisioned that the proximal and distal ends of balloon 24 may each be attached to the outer surface of outer shaft 18 using an adhesive or a bond, such as, for example, a heat bond. In some embodiments, the proximal and distal ends of balloon 24 may each be attached to the outer surface of outer shaft 18 by a coupling device, such as, for example, a ring or collar that surrounds each of the proximal and distal ends of balloon 24 such that the proximal and distal ends of balloon 24 are each positioned between a ring or collar and the outer surface of outer shaft 18. The ring or collar can be radiopaque and can be used to determine the position of the first or second balloon using x-ray detection means during the surgical procedure. In one embodiment, balloon 24 is integrated into outer shaft 18. In one embodiment, balloon 24 is integrated on outer shaft 18.

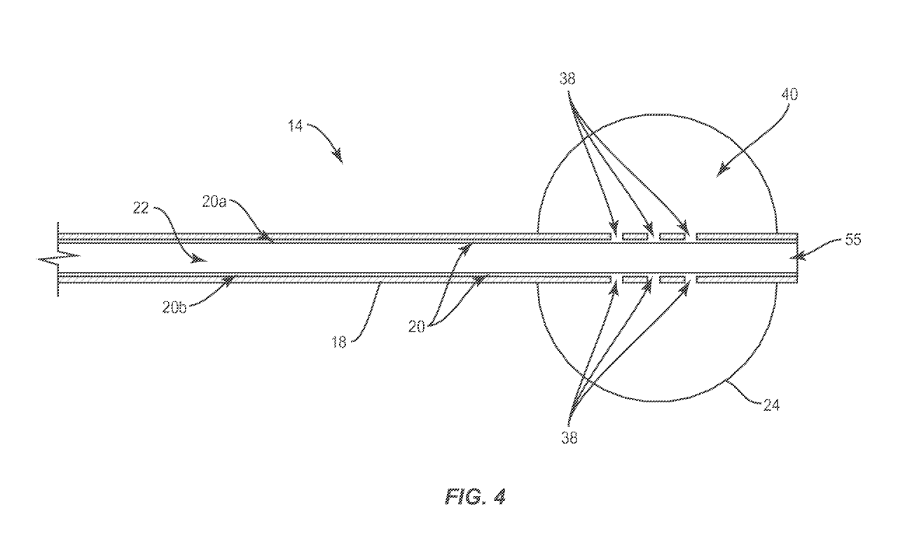

Outer shaft 18 comprises one of a plurality of apertures 38, as shown in FIG. 4. Apertures 38 extend through the outer surface of outer shaft 18 and an opposite inner surface of outer shaft 18 that defines first lumen 20. Apertures 38 are each in communication with first lumen 20 and an interior cavity 40 of balloon 24 such that an inflation material can move from the material source, through passageway 32, first lumen 20 and apertures 38 and into interior cavity 40 to move balloon 24 from an uninflated or unexpanded configuration to an inflated or expanded configuration. First lumen 20 is closed at a distal end of outer shaft 18, as shown in FIG. 4, for example. That is, first lumen 20 does not extend through a distal end surface of outer shaft 18 that extends transverse or perpendicular to longitudinal axis L to prevent inflation material from escaping first lumen 20. In embodiments in which first lumen 20 comprises first section 20a and second section 20b, outer shaft 18 comprises one or a plurality of apertures 38 that are in communication with interior cavity 40 and first section 20a and one or a plurality of apertures 38 that are in communication with interior cavity 40 and second section 20b, as shown in FIGS. 4, 5A and 5B. In some embodiments, apertures 38 may have various cross section configurations, such as, for example, oval, oblong, triangular, rectangular, square, polygonal, irregular, uniform, non-uniform, variable, tubular and/or tapered. In some embodiments, apertures 38 may be disposed at alternate orientations, relative to longitudinal axis L, such as, for example, transverse, perpendicular and/or other angular orientations such as acute or obtuse and/or may be offset or staggered.

In some embodiments, balloon 24 comprises a material that is resistant to bone cement, such as, for example, PMMA, such as, for example, nylon. That is, balloon 24 comprises a material that will resist degradation when in contact with bone cement before and after the bone cement hardens or polymerizes. In some embodiments, balloon 24 comprises a material that degrades when in contact with bone cement and is coated with a material that will resist degradation when in contact with bone cement before and after the bone cement hardens or polymerizes to prevent balloon 24 from degrading when in contact with bone cement.

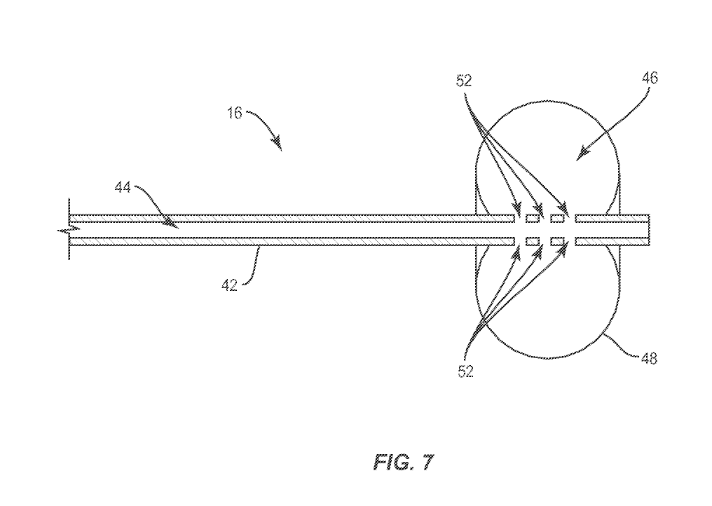

Second inflatable bone tamp 16 comprises an inner shaft 42. Inner shaft 42 has a maximum diameter that is less than that of second lumen 22 of outer shaft 18 such that inner shaft 42 can be slidably positioned within second lumen 22. Inner shaft 42 is removable from second lumen 22 such that first inflatable bone tamp 14 can be separated from second inflatable bone tamp 16, as shown in FIG. 2. Inner shaft 42 defines a third lumen 44 (FIG. 7) that is in communication with an interior cavity 46 of a second expandable body, such as, for example, a balloon 48 that is coupled to a distal end of inner shaft 42. Inner shaft 42 comprises an opening 50 in a proximal end of inner shaft 42 that is in communication with third lumen 44, as shown in FIG. 2.

Balloon 48 extends between a proximal end and an opposite distal end. The proximal and distal ends of balloon 48 are each coupled to an outer surface of inner shaft 42. It is envisioned that the proximal and distal ends of balloon 48 may each be attached to the outer surface of inner shaft 42 using an adhesive or a bond, such as, for example, a heat bond. In some embodiments, the proximal and distal ends of balloon 48 may each be attached to the outer surface of inner shaft 42 by a coupling device, such as, for example, a ring or collar that surrounds each of the proximal and distal ends of balloon 48 such that the proximal and distal ends of balloon 48 are each positioned between a ring or collar and the outer surface of inner shaft 42.

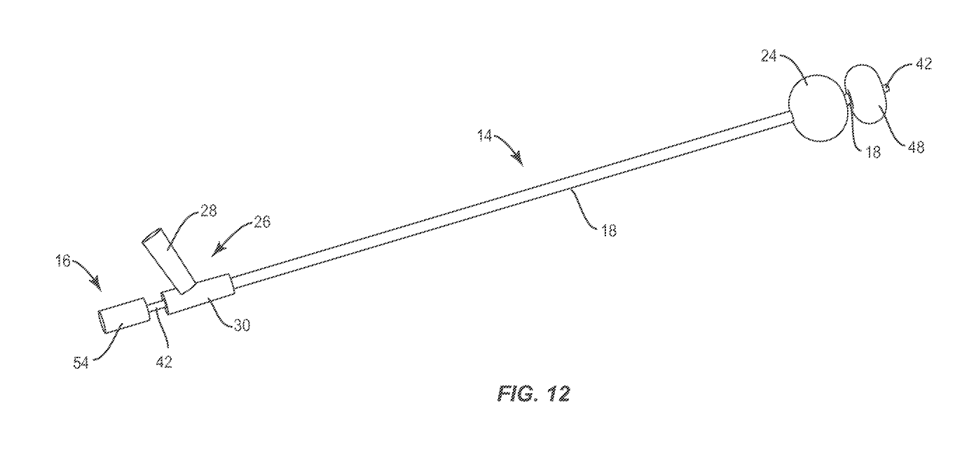

Inner shaft 42 comprises one or a plurality of apertures 52, as shown in FIG. 7. Apertures 52 each extend through the outer surface of inner shaft 42 and an opposite inner surface of inner shaft 42 that defines third lumen 44. Apertures 52 are each in communication with third lumen 44 and interior cavity 46 of balloon 48 such that an inflation material can move from a material source, through opening 50, third lumen 44 and apertures 52 and into interior cavity 46 of balloon 48 to move balloon 48 from an uninflated or unexpanded configuration to an inflated or expanded configuration. In some embodiments, balloon 48 engages a distal end surface of outer shaft 18 when balloon 48 is in the inflated or expanded configuration, as shown in FIG. 12, for example. Third lumen 44 is closed at a distal end of inner shaft 42, as shown in FIG. 7, for example. That is, third lumen 44 does not extend through a distal end surface of inner shaft 42 that extends transverse or perpendicular to a longitudinal axis defined by inner shaft 42 to prevent inflation material from escaping third lumen 44. In some embodiments, apertures 52 may have various cross section configurations, such as, for example, oval, oblong, triangular, rectangular, square, polygonal, irregular, uniform, non-uniform, variable, tubular and/or tapered. In some embodiments, apertures 52 may be disposed at alternate orientations, relative to the longitudinal axis defined by inner shaft 42, such as, for example, transverse, perpendicular and/or other angular orientations such as acute or obtuse and/or may be offset or staggered.

Inner shaft 42 has a maximum length defined by the distance between the proximal and distal ends of inner shaft 42 that is greater than a maximum length of outer shaft 18 that is defined by the distance between the proximal and distal ends of outer shaft 18 such that balloon 48 may extend through an opening 55 (FIG. 4) in a distal end of outer shaft 18 to position balloon 48 distal to balloon 24, as shown in FIG. 1. The proximal end of inner shaft 42 comprises an enlarged handle 54 configured to facilitate gripping of inner shaft 42. Handle 54 has an outer diameter that is greater than a diameter of channel 34 of second extension 30 such that handle 54 is prevented from entering channel 34 as inner shaft 42 slides within channel 34 and/or second lumen 22. In some embodiments, handle 54 comprises threads configured to mate with threads of a material source to couple the material source to handle 54. In some embodiments, the material source can be variously connected with handle 54, such as, for example, monolithic, integral connection, frictional engagement, threaded engagement, mutual grooves, screws, adhesive, nails, barbs and/or raised element.

It is envisioned that the shapes and sizes of balloon 24 and/or balloon 48 when in the inflated or expanded configuration can be selected to provide a desired result during a procedure. For example, balloon 24 and/or balloon 48 may include shapes such as spheres, cylinders, etc. and have different dimensions to make balloon 24 and/or balloon 48 narrower or wider in a longitudinal direction, or extend further in a radial direction. In some embodiments, balloon 24 and/or balloon 48 comprises a compliant material or non-compliant material. In some embodiments, balloon 24 comprises a compliant material and balloon 48 comprises a non-compliant material. In some embodiments, balloon 24 and balloon 48 each comprise a compliant material. In some embodiments, balloon 24 has a surface area that is greater than a surface area of balloon 48 when balloons 24, 48 are in the inflated or expanded configurations to achieve lordosis, for example. In some embodiments, balloon 24 has a surface area that is less than a surface area of balloon 48 when balloons 24, 48 are in the inflated or expanded configurations to achieve lordosis, for example. In some embodiments, balloon 24 has a maximum width or diameter in a direction that is perpendicular to longitudinal axis L that is greater than a maximum width or diameter of balloon 48 in a direction that is perpendicular to a longitudinal axis defined by inner shaft 42 when balloons 24, 48 are in the inflated or expanded configurations to achieve lordosis, for example. In some embodiments, balloon 24 has a maximum width or diameter in a direction that is perpendicular to longitudinal axis L that is less than a maximum width or diameter of balloon 48 in a direction that is perpendicular to a longitudinal axis defined by inner shaft 42 when balloons 24, 48 are in the inflated or expanded configurations to achieve lordosis, for example. In some embodiment, balloon 24 has a different size than balloon 48 such that balloons 24, 48 present an angled surface for the repair of bones that also comprise angled or sloped surfaces.

It is envisioned that balloon 24 and/or balloon 48 can be a single or multi-layered balloon where each balloon layer has the same diameter and/or wall thickness, is comprised of the same material or materials having substantially identical mechanical properties, and has the same degree of molecular orientation in the body portion of the balloon. It will be apparent that in some situations it will be desirable to have some balloon layers having different thicknesses, materials, and/or degree of molecular orientations upon deflation, while at the same time having equivalent size, mechanical properties, and/or orientation upon inflation or expansion. For other applications, it will be apparent that one can vary size, material, and/or orientation to at least some degree, depending upon the requirements of a particular application. In embodiments wherein balloon 24 is a multi-layered balloon, balloon 24 comprises at least one inner layer comprising a material that degrades when in contact with bone cement and an outer layer that will resist degradation when in contact with bone cement before and after the bone cement hardens or polymerizes to prevent balloon 24 from degrading when in contact with bone cement.

It is contemplated that balloon 24 may include an impenetrable structural layer having low friction surfaces so as to facilitate deployment through a delivery tube, such as, for example, a cannula and prevent rupture of balloon 24 as it is inflated or expanded in situ. Likewise, balloon 48 may include an impenetrable structural layer having low friction surfaces so as to facilitate deployment through second lumen 22 and prevent rupture of balloon 48 as it is inflated or expanded in situ. Further variations are contemplated involving different combinations of lubricating layers and structural layers. In some embodiments, structural layers of balloon 24 and/or balloon 48 can contain polyamides, polyesters, polyethylenes, polyurethanes, their co-polymers and combinations thereof.

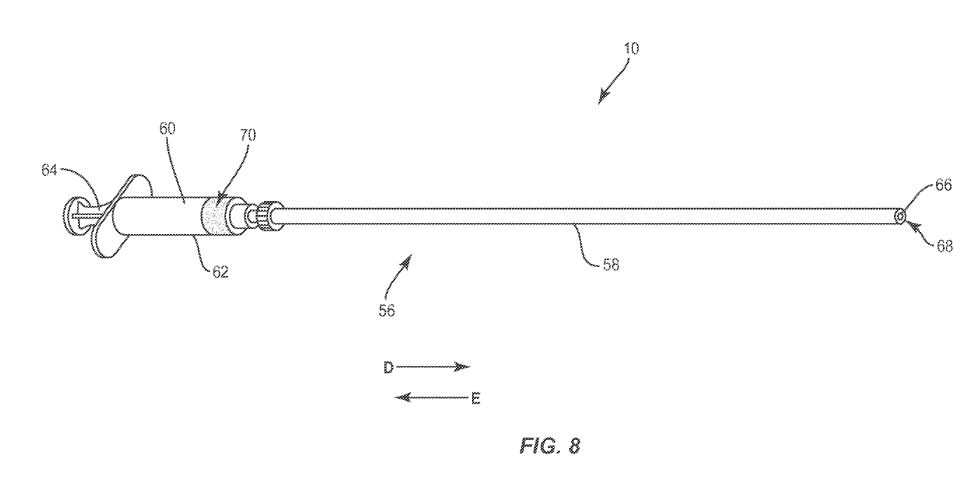

In some embodiments, system 10 comprises instruments that are configured to be movably disposed in second lumen 22 of outer shaft 18 when inner shaft 42 is removed from outer shaft 18. For example, system 10 may comprise one or a plurality of instruments 56 shown in FIG. 8. Instrument 56 may be a cement delivery device, an aspiration device and/or a suction device. Instrument 56 comprises a tube 58 that is coupled to a syringe 60, as shown in FIG. 8. Syringe 60 comprises a body 62 that defines a cavity configured for disposal of a material 70. Syringe 60 comprises a plunger 64 movably disposed in the cavity of syringe 60. In one embodiment, plunger 64 is configured to move relative to body 62 in the direction shown by arrow D in FIG. 8 to push material 70 through the cavity of syringe 60 and into a passageway 66 of tube 58. Material moves through passageway 66 and out of a hole 68 in a distal end of tube 58 that is in communication with passageway 66. In one embodiment, plunger 64 is configured to be moved in the direction shown by arrow E in FIG. 8 to create suction such that material 70 positioned adjacent hole 68 is sucked into hole 68. The suction created by plunger 64 moves material 70 into passageway 66. Material 70 moves from passageway 66 and into the cavity defined by body 62 of syringe 60.

In some embodiments, material 70 is a material used in a surgical procedure, such as, for example, inflation material, aspiration material and/or bone cement. In some embodiments, material 70 is waste material, such as, for example, material that is intended to be removed from a patient's anatomy (bone chips, bone fragments, blood clots, inorganic or man-made material, etc.). In embodiments wherein instrument 56 is used to deliver bone cement and material 70 is a two part bone cement, it is envisioned that body 62 may define two cavities that are spaced apart from one another by a wall of body 62. One part of the bone cement can be disposed in one of the cavities and the other part of the bone cement can be disposed in the other cavity such that moving plunger 64 in the direction shown by arrow D in FIG. 12 moves the both parts of the bone cement into passageway 66 where the parts mix with one another. In embodiments wherein material 70 is a two part bone cement, it is envisioned that one part of the bone cement is a prepolymer powder and the other part of the bone cement is a liquid monomer.

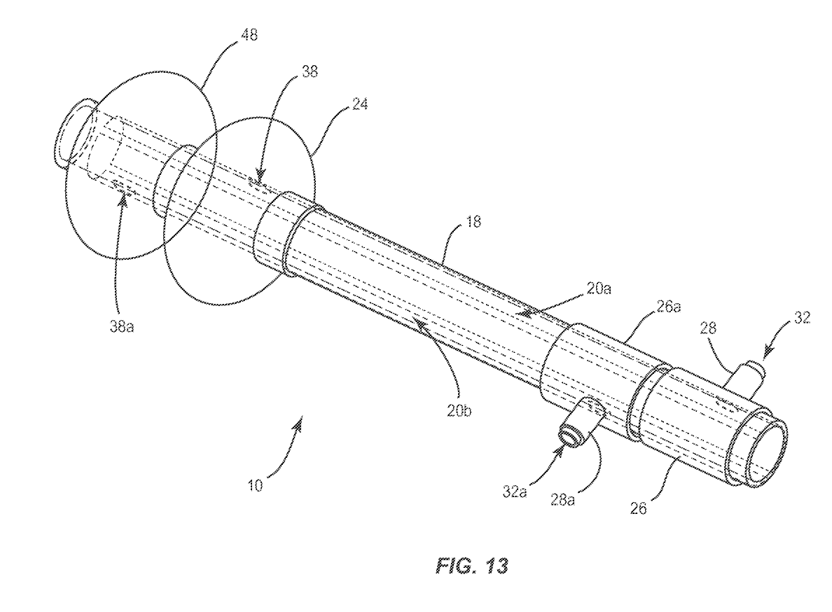

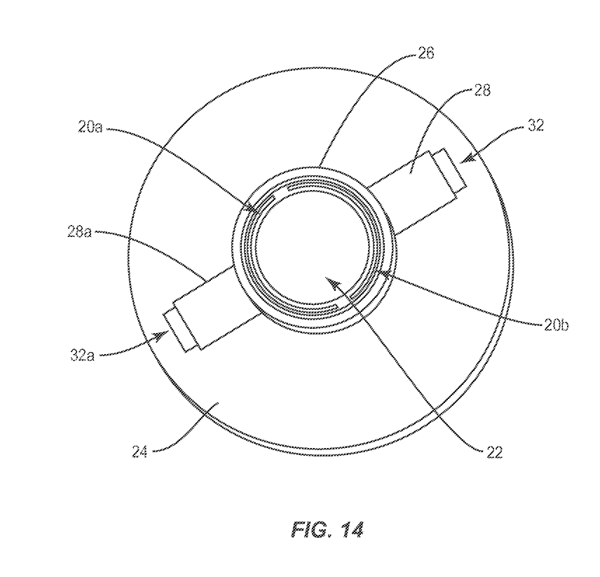

In one embodiment, shown in FIGS. 13 and 14, balloon 24 and balloon 48 are both coupled to outer shaft 18. First lumen 20 comprises first section 20a and second section 20b similar to that shown in FIGS. 3-5B. Outer shaft 18 comprises connector 26 and a second connector 26a. Passageway 32 of connector 26 is in communication with first section 20a such that when a material source is coupled to connector 26, the material source may deliver inflation material through passageway 32, into first section 20 of first lumen 20, through aperture 38 and into interior cavity 40 of balloon 24 to move balloon 24 from an uninflated or unexpanded configuration to an inflated or expanded configuration. A passageway 32a of second connector 32a is in communication with second section 20b such that when a material source is coupled to connector 26a, the material source may deliver inflation material through passageway 32a, into second section 20b of first lumen 20, through an aperture 38a in a distal end of outer shaft 18 and into interior cavity 46 of balloon 48 to move balloon 48 from an uninflated or unexpanded configuration to an inflated or expanded configuration.

The configuration of the IBT shown in FIGS. 13 and 14 allows balloons 24, 48 to be introduced to a surgical site simultaneously and inflated (and/or deflated) independently of one another. It is envisioned that a separate IBT, similar to second IBT 16 shown in FIGS. 2 and 7, may be removably positioned within second lumen 22 of the IBT shown in FIGS. 13 and 14 to provide a surgeon with a greater number of options with regard to the number and types of voids that can be created and at least partially filed using system 10.

In assembly, operation and use, system 10 is employed with a surgical procedure, such as, for a correction or treatment of bone fractures. It is contemplated that one or all of the components of system 10 can be delivered or implanted as a pre-assembled device or can be assembled in situ. System 10 may be completely or partially revised, removed or replaced as part of the operation. System 10 can be employed with a surgical correction treatment of an applicable condition or injury of an affected portion of a vertebra, a calcaneus bone, bones of the hands, bones of the arms and legs, etc. and other areas within a body. System 10 can also be used to distract joints for ligament repair or distract soft tissue.

In use, to treat a bone defect, such as, for example, a vertebral compression fracture, a medical practitioner obtains access to a surgical site including the fractured vertebra in any appropriate manner, such as through incision and retraction of tissues. In one embodiment, a drill is employed to remove bone tissue to provide access to a repair site. It is envisioned that system 10 can be used in any existing surgical method or technique including open surgery, mini-open surgery, minimally invasive surgery and percutaneous surgical implantation, whereby the fractured or injured bone is accessed through a mini-incision or sleeve that provides a protected passageway to the area. Once access to the surgical site is obtained, the particular surgical procedure can be performed for treating the injury or disorder. The configuration and dimension of system 10 is determined according to the configuration, dimension and location of a selected section of the bone fracture and the requirements of a particular application.

An incision is made in the body of a patient and a cutting instrument (not shown) creates a surgical pathway for implantation of components of system 10. This may include the use of a cannula or other device. A preparation instrument (not shown) can be employed to prepare tissue surfaces, as well as for aspiration and irrigation of a surgical region according to the requirements of a particular surgical application.

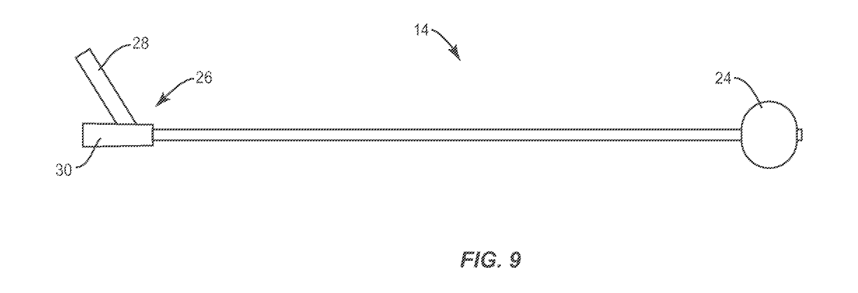

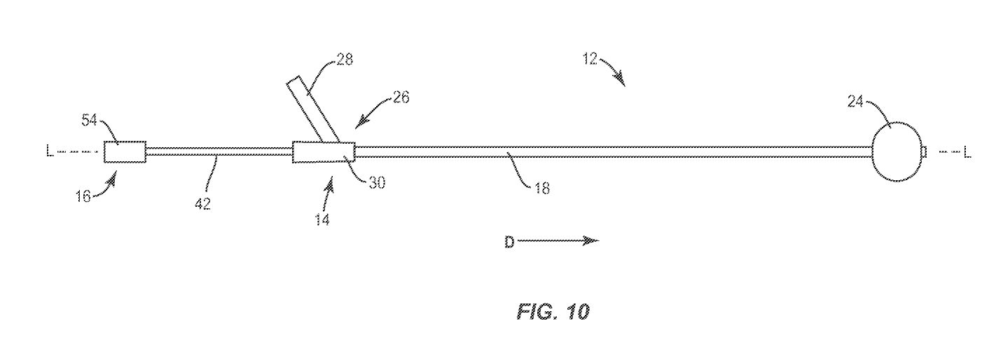

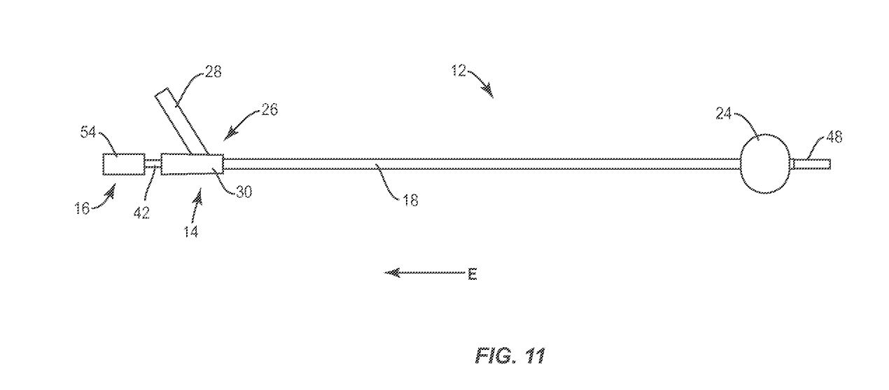

First inflatable bone tamp 14, shown in FIG. 9, for example, may be inserted through the incision such that balloon 24 is positioned adjacent to the surgical site. In some embodiments, balloon 24 may be positioned within a vertebral body of the fractured vertebra. Second inflatable bone tamp 16 may be inserted through first inflatable bone tamp 14 by inserting inner shaft 42 through opening 36 in outer shaft 18 and into second lumen 22 of outer shaft 18, as shown in FIG. 10. Second inflatable bone tamp 16 may be translated axially along longitudinal axis L in the direction shown by arrow D in FIG. 10 to move balloon 48 through opening 55 in outer shaft 18 such that balloon 48 is positioned distal to balloon 24, as shown in FIG. 11. In some embodiments, balloon 48 is positioned within the vertebral body of the fractured vertebra when balloon 48 is positioned distal to balloon 24. It is envisioned that second inflatable bone tamp 16 may be inserted within first inflatable bone tamp 14 to position balloon 48 a selected distance from balloon 24. In some embodiments, the selected distance corresponds to the size of the vertebral body. It is further envisioned that balloons 24, 48 may each have a surface area that is selected based upon the size of the vertebral body.

A material source, such as, for example, a source of compressed air or saline may be removably coupled to first extension 28 such that an inflation material can move from the material source, through passageway 32, first lumen 20 and apertures 38 and into interior cavity 40 of balloon 24 to move balloon 24 from an uninflated or unexpanded configuration to an inflated or expanded configuration. In embodiments where first lumen 20 includes first section 20a and second section 20b, the material source is coupled to first extension 28 such that an inflation material can move from the material source, through passageway 32, first section 20a and apertures 38 and into interior cavity 40 of balloon 24 and a second material source is coupled to third extension 28a such that an inflation material can move from the second material source, through passageway 32a, second section 20b and apertures 38 and into interior cavity 40 of balloon 24. In some embodiments, inflation material is moved simultaneously through first and second sections 20a, 20b to inflate balloon 24. In some embodiments, the material source is first coupled to first extension 28 to fill interior cavity 40 through first section 20a of first lumen 20 and the material source is then removed from first extension 28 and coupled to third extension to fill interior cavity 40 through second section 20b of first lumen 20. As balloon 24 moves from the uninflated or unexpanded configuration to the inflated or expanded configuration, balloon 24 creates a first void in the vertebral body, reduces the fracture and obtains at least some degree of height restoration.

The same material source that was coupled to first extension 28 or another material source may be removably coupled to second extension 30 such that an inflation material can move from the material source, through opening 50 of inner shaft 42, third lumen 44 and apertures 52 and into interior cavity 46 of balloon 48 to move balloon 48 from an uninflated or unexpanded configuration shown in FIG. 11 to an inflated or expanded configuration shown in FIG. 12. As balloon 48 moves from the uninflated or unexpanded configuration to the inflated or expanded configuration, balloon 48 creates a second void in the vertebral body, reduces the fracture and obtains at least some degree of height restoration. In some embodiments, balloons 24, 48 contact one another when balloons 24, 48 are in the inflated or expanded configurations, as shown in FIG. 1, such that the first and second voids created by balloons 24, 48 overlap. In some embodiments, balloons 24, 48 are spaced apart from one another when balloons 24, 48 are in the inflated or expanded configurations, as shown in FIG. 12, such that the first and second voids are also spaced apart from one another.

After the first and second voids are created by balloons 24, 48, balloon 48 may be moved from the inflated or expanded configuration to the uninflated or unexpanded configuration by removing or withdrawing the inflation material from interior cavity 46 of balloon 48. In some embodiments, the inflation material is removed from interior cavity 46 by providing suction within first lumen 20 of outer shaft 18 using the material source or a suction device such that the inflation material moves out of interior cavity 40 through apertures 38 and into first lumen 20. Balloon 48 may remain in the inflated or expanded configuration after balloon 48 is moved to the uninflated or unexpanded configuration, as shown in FIG. 11, to maintain the height restoration provided by balloon 24 and/or balloon 48.

Second inflatable bone tamp 16 may be removed from first inflatable bone tamp 14 by translating inner shaft 42 relative to outer shaft 18 in the direction shown by arrow E in FIG. 11 until balloon 48 moves through opening 36 in outer shaft 18 and first inflatable bone tamp 14 is separated from second inflatable bone tamp 16, as shown in FIG. 2. A cement delivery tool, such as, for example, instrument 56 may be inserted into second lumen 22 and translated relative to outer shaft 18 in the direction shown by arrow D in FIG. 8 until a distal tip of tube 58 extends through opening 55 in outer shaft 18. In some embodiments, the distal tip of tube 58 is positioned such that the distal tip of tube 58 is positioned within or adjacent to the second void created by balloon 48. Bone cement, such as, for example, material 70 may be injected into the second void using instrument 56 to fill the second void created by balloon 48. In some embodiments, balloon 48 may remain in the inflated or expanded configuration while the bone cement hardens or cures within the second void, to maintain the height restoration provided by balloon 24 and/or balloon 48. In some embodiments, instrument 56 may be removed from first inflatable bone tamp 14 after the second void is filled with bone cement. First inflatable bone tamp 14 may be removed from the patient through the incision after the bone cement hardens or polymerizes within the second void to maintain the height restoration created by balloon 48. In some embodiments, balloon 24 may be moved from the inflated or expanded configuration to the uninflated or unexpanded configuration by removing or withdrawing the inflation material from interior cavity 40 of balloon 24 prior to removing first inflatable bone tamp 14 from the patient.

In some embodiments, a bone cement delivery tool, such as, for example, instrument 56 is inserted through the incision such that the distal tip of tube 58 is positioned within or adjacent to the first void created by balloon 24 after first inflatable bone tamp 14 is removed from the patient. Bone cement, such as, for example, material 70 may be injected into the first void using instrument 56 to fill the first void created by balloon 24. Instrument 56 may then be removed from the patient through the incision.

Other components of system 10 may delivered to the surgical site. For example, in some embodiments, a suction device, such as, for example, instrument 56 may be inserted through second lumen 22 such that a distal tip of tube 58 extends through opening 55 in outer shaft 18. In some embodiments, the distal tip of tube 58 is positioned within or adjacent to the second void created by balloon 48 after the second inflatable bone tamp is removed from the patient and before the second void is filled with bone cement. Instrument 56 may be used to create suction as discussed herein to remove debris, such as, for example, bone chips or waste material 70 from the second void. In some embodiments, the distal tip of tube 58 is positioned within or adjacent to the first void created by balloon 24 after first inflatable bone tamp 14 is removed from the patient and before the second void is filled with bone cement. Instrument 56 may be used to create suction as discussed herein to remove debris, such as, for example, bone chips or waste material 70 from the first void.

In some embodiments, an aspiration device, such as, for example, instrument 56 may be inserted through second lumen 22 such that a distal tip of tube 58 extends through opening 55 in outer shaft 18. In some embodiments, the distal tip of tube 58 is positioned within or adjacent to the second void created by balloon 48 after the second inflatable bone tamp is removed from the patient and before the second void is filled with bone cement. Instrument 56 may be used to aspirate the second void as discussed herein. In some embodiments, the distal tip of tube 58 is positioned within or adjacent to the first void created by balloon 24 after first inflatable bone tamp 14 is removed from the patient and before the second void is filled with bone cement. Instrument 56 may be used to aspirate the first void as discussed herein.

In one embodiment, system 10 includes an agent, which may be disposed, packed or layered within, on or about the components and/or surfaces of system 10, such as, for example, on an outer surface of balloon 24 and/or an outer surface of balloon 48. It is envisioned that the agent may include bone growth promoting material, such as, for example, bone graft to enhance fixation of the fixation elements with the bone in need of repair.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of system 10 can be made of radiolucent materials such as polymers. Radiomarkers may be included for identification under x-ray, fluoroscopy, CT or other imaging techniques. For example, the tip of the stylet can be used as a visualization marker replacing the distal marker band. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of system 10. Upon completion of the procedure, the surgical instruments and assemblies are removed. The incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto. The embodiments above can also be modified so that some features of one embodiment are used with the features of another embodiment. One skilled in the art may find variations of these preferred embodiments, which, nevertheless, fall within the spirit of the present invention, whose scope is defined by the claims set forth below.

What is claimed is:

1. A bone tamp comprising:
an outer shaft defining a first lumen and a second lumen;
a first expandable body coupled to the outer shaft, the first expandable body being in communication with the first lumen such that an inflation material may be moved through the first lumen and into the first expandable body;
an inner shaft movably positioned within the second lumen, the inner shaft defining a third lumen; and
a second expandable body coupled to the inner shaft, the second expandable body being in communication with the third lumen such that an inflation material may be moved through the third lumen and into the second expandable body.

2. A bone tamp as recited in claim 1, wherein the first lumen is isolated from the second lumen.

3. A bone tamp as recited in claim 1, wherein the first lumen surrounds the second lumen.

4. A bone tamp as recited in claim 1, wherein the first expandable body is inflatable independent of the second expandable body.

5. A bone tamp as recited in claim 1, wherein the inner shaft is removable from within the second lumen.

6. A bone tamp as recited in claim 1, wherein the first expandable body extends between a first end and an opposite second end, the first and second ends each being coupled to an outer surface of the outer shaft, the outer shaft comprising an opening that extends through the outer surface and is in communication with the first lumen, the opening being positioned between the first and second ends such that an inflation material may be moved through the first lumen and the opening and into the first expandable body to move the first expandable body from an unexpanded configuration to an expanded configuration.

7. A bone tamp as recited in claim 1, wherein the first expandable body is operable independently of the second expandable body.

8. A bone tamp as recited in claim 1, wherein the first expandable body has a larger surface area than that of the second expandable body when the first and second expandable bodies are each in an expanded configuration.

9. A bone tamp as recited in claim 1, wherein the first expandable body is spaced apart from the second expandable body when the first and second expandable bodies are each in an expanded configuration.

10. A bone tamp as recited in claim 1, wherein the inner shaft has a maximum length that is greater than that of the outer shaft.

11. A bone tamp as recited in claim 1, wherein the first expandable body is formed from a material that is resistant to poly(methyl methacrylate).

12. A bone tamp as recited in claim 1, wherein the first expandable body is formed from a compliant material and the second expandable body is formed from a non-compliant material.

13. A bone tamp as recited in claim 1, wherein the first and second expandable bodies are each formed from a compliant material.

14. A surgical system comprising:
the balloon tamp recited in claim 1;
a bone cement delivery device; and
bone cement,
wherein the inner shaft is removably positioned within the second lumen and the second lumen is configured for disposal of at least a portion of the bone cement delivery device such that the bone delivery device can deliver the bone cement from a distal end of the inner shaft.

15. A method of treating a bone defect, comprising:
providing the bone tamp recited in claim 1;
positioning the first expandable body within a bony structure and injecting an inflation material through the first lumen and into the first expandable body to move the first expandable body from an unexpanded configuration to an expanded configuration;
positioning the second expandable body within the bony structure and injecting an inflation material through the third lumen and into the second expandable body to move the second expandable body from an unexpanded configuration to an expanded configuration such that the second expandable body creates a void within the bony structure;
withdrawing the inflation material from the second expandable body to move the second expandable body from the expanded configuration to an unexpanded configuration;
removing the inner shaft from the second lumen; and
at least partially filling the void by injecting bone cement through the second lumen into the void.

16. A method as recited in claim 15, wherein the first expandable body is in the expanded configuration while the void is at least partially filled with bone cement.

17. A method as recited in claim 15, wherein:
moving the first expandable body to the expanded configuration creates a second void in the bony structure; and
the method further comprises:
withdrawing the inflation material from the first expandable body to move the first expandable body from the expanded configuration to an unexpanded configuration after at least partially filling the void; and
at least partially filling the second void by injecting bone cement from the bone cement delivery device and into the second void.

18. A method as recited in claim 15, further comprising:
inserting a suction device into the second lumen prior to inserting the bone cement delivery device into the second lumen;
removing material within the void using the suction device.

19. A method as recited in claim 15, further comprising:
moving the inner shaft relative to the outer shaft to space the second expandable body apart a selected distance from first expandable body, the selected distance corresponding to a size of the bony structure,
wherein a surface area of each of the expandable bodies is based on the size of the bony structure.

20. A surgical system, comprising:
a bone tamp comprising:
an outer shaft defining a first lumen and a second lumen, the first lumen surrounding the second lumen such that the first lumen is isolated from the second lumen,
a first expandable body coupled to the outer shaft, the first expandable body being in communication with the first lumen such that an inflation material may be moved through the first lumen and into the first expandable body,
an inner shaft slidably positioned within the second lumen, the inner shaft defining a third lumen, the inner shaft being removable from the outer shaft, and
a second expandable body coupled to the inner shaft, the second expandable body being in communication with the third lumen such that an inflation material may be moved through the third lumen and into the second expandable body;
a bone cement delivery device; and
bone cement,
wherein the inner shaft is removably positioned within the second lumen and the second lumen is configured to carry the bone cement out of the distal end of the second lumen,
wherein the first expandable body is inflatable independent of the second expandable body, and
wherein the first expandable body has a surface area that is greater than that of the second expandable body when the first and second expandable bodies are each in an expanded configuration.
